(19) United States
(12) Patent Application Publication
(10) Pub. No.: US 2016/0206178 A1
(43) Pub. Date: Jul. 21, 2016

(54) SURGICAL APPARATUSES FOR COUPLING ELONGATED MEMBERS TO ENDOSCOPES, AND RELATED METHODS

(71) Applicant: SCOTT & WHITE HEALTHCARE, Temple, TX (US)

(72) Inventor: Melvin LAU, Round Rock, TX (US)

(21) Appl. No.: 14/913,199

(22) PCT Filed: Aug. 20, 2014
(86) PCT No.: PCT/US2014/051910

§ 371 Date: Feb. 19, 2016

Related U.S. Application Data
(60) Provisional application No. 61/868,003, filed on Aug. 20, 2013.

(51) Int. Cl. A61B 1/00

(52) U.S. Cl. CPC A61B 1/0014; A61B 1/00137; A61B 1/0011

(57) ABSTRACT

Surgical apparatuses that include one or more structures that can be used to couple an elongated member, such as surgical tubing, to an endoscope, to yield a channel external of the endoscope that can be used to remove material from or deliver material to a desired location within a subject, like a human patient. Methods of using such surgical apparatuses are also disclosed.

### BACKGROUND

This application claims the benefit of priority to U.S. Provisional Patent Application Ser. No. 61/868,003, filed August 2013, hereby incorporated by reference in its entirety.

### FIELD OF THE INVENTION

The invention relates generally to the delivery and/or suction of matter to desired anatomical locations and more particularly, in some respects, to medical devices (e.g., surgical apparatuses) that include an elongated device having at least one passageway (e.g., a tube) attachable via a coupling structure to an endoscope and useful for suctioning and/or inserting material during a procedure (e.g., a surgical procedure).

### DESCRIPTION OF RELATED ART

Endoscopes are typically used by physicians to inspect the interior of a body of a patient. An endoscope may include one or more channels to allow entry of medical instruments or manipulators, and for removal and introduction of contents. The rate at which contents can be removed from or introduced to a patient's interior is limited by the diameter of the relevant channel. Additionally, some contents, such as stomach or colon contents, may be too large to pass through a normal endoscope internal channel.

### SUMMARY

Disclosed are surgical apparatuses for coupling an elongated member, like surgical tubing, to an endoscope. Some embodiments of the present surgical apparatuses include an elongated member, such as a flexible tube, and a coupling structure configured to couple the elongated structure to an endoscope. The elongated structure has a channel that extends between first and second openings in the elongated structure, which openings may, in some embodiments, be located at the distal and proximal ends of the elongated structure. The coupling structure may be attached to the elongated structure, such as with an adhesive, or may be formed integrally with the elongated structure. The coupling structure may, in some embodiments, comprise a cap configured to fit over a distal end of an endoscope. The coupling structure may, in some embodiments, include curved portions that extend toward but do not touch each other. The coupling structure may, in some embodiments, be configured to not completely surround a tubular structure (such as an endoscope) when coupled to the tubular structure. The elongated structure may, in other embodiments, comprise a flexible sleeve configured to fit over both an endoscope and the elongated member, and may be configured to be unrolled following placement over both structures; the flexible sleeve may be provided in a rolled-up form. The elongated member may have an inner surface that defines its channel, and the channel may have any suitable cross-sectional profile at any location along its length (which profile may be defined at a given location by the intersection of the inner surface and a plane oriented perpendicular to an axis centered in and running within the channel), such profile having a cross sectional area of at least 0.12 centimeters squared (cm²) in some embodiments, and, in more specific embodiments, a cross sectional area of at least 0.28 cm²; such profile may exist at a location along (or at several spaced-apart locations along) the length of the channel, or may exist along the length of the channel. In some embodiments, the surgical apparatus also includes another coupling structure, such as at least one plastic tie (and, in some embodiments, multiple plastic ties), that can be used to couple the elongated member to an endoscope at a more proximal location than the above-mentioned coupling structure. In some embodiments, the surgical apparatus also includes a hub, that can have a male end over which the proximal end of the elongated member may be positioned to couple the elongated member to the hub, the hub being equipped with a valve; additionally or alternative, the surgical apparatus includes a clamp that can be positioned at a location along the elongated member and used to inhibit flow through the elongated member as desired.

Some embodiments of the present surgical apparatuses include an elongated member coupling structure (such as a cap or a sleeve) configured to be coupled to and surround an exterior portion of an elongated member; and a coupling structure attached to or integral with the elongated member coupling structure and configured to couple the elongated member coupling structure to an endoscope.

The surgical apparatus may comprise a kit that includes a container (such as a flexible pouch), one or more of the components disclosed, and instructions for use (which may be included on the outside of the container (e.g., via a label) or on material (e.g., a card or other insert) disposed inside the container).

Also disclosed are methods of using a surgical apparatus, including methods of using the apparatus during a medical procedure, and methods of instructing another or others (e.g., through a demonstration that is live and in person, live and remote, or pre-recorded, and which may be a mock demonstration) on using the apparatus. Some embodiments of the present methods, which may be characterized as a medical procedure-related methods, comprise coupling an elongated member having a distal end to an endoscope having a distal end so that the distal ends of the elongated member and of the endoscope are aligned. Some embodiments of the present methods comprise coupling an elongated member having a length and a channel to an endoscope having a length so that: the elongated member is positioned beside the endoscope for a portion of their respective lengths, no portion of the elongated member is coaxial with any portion of the endoscope, and no portion of the channel of the elongated member is in communication with any channel of the endoscope. The elongated member of any of these embodiments may be any of the disclosed elongated members, including any of those discussed above in this Summary section. In some embodiments, the coupling may be accomplished with at least a coupling structure, such as a cap configured to fit over the end of the endoscope, that is attached to or integral with the elongated member. In other embodiments, the coupling may be accomplished with at least a coupling structure comprising a flexible sleeve that is positioned around both the endoscope and the elongated member. In still other embodiments, the coupling may be accomplished using at least a coupling structure to which the elongated member is coupled (e.g., via an elongated member coupling structure) and to which the endoscope is coupled. In some embodiments, the method may include using at least another coupling structure to further couple the elongated member to the endoscope at a location proximal of the first coupling structure; the other coupling structure may comprise at least one plastic tie, and the use of the plastic tie to achieve the further coupling may include wrapping the tie around both the endoscope and the elongated member. In some embodiments, the method may also include coupling the elongated member (e.g., its proximal end) to a suction (or vacuum) source (such as a suction canister) or to a fluid-advancing source (such as a syringe or a pump), inserting the endoscope and the elongated member into an animal (e.g., a human), and using the channel of the elongated member to introduce material into or remove material from the animal, which use may involve the suction source or the fluid-advancing source. Some embodiments of the present methods may also include inserting an endoscope coupled, in one of the manners set forth herein, to an elongated member into any location (e.g., a lumen, like a body cavity, or extra-lumen, like the pleural cavity or the peritoneum) of a subject (e.g., a human patient), including a stomach, colon, intestine, bladder, bronchus, ear, nose, throat, or the like.

The term “coupled” is defined as connected, although not necessarily directly, and not necessarily mechanically. The terms “a” and “an” are defined as one or more unless this disclosure explicitly requires otherwise. The term “substantially” is defined as largely but not necessarily wholly what is specified (and includes what is specified), as understood by a person of ordinary skill in the art. In any disclosed embodiment, the terms “substantially,” “approximately,” and “about” may be substituted with “within [a percentage] of” what is specified, where the percentage includes 0.1, 1, 5, and 10 percent.

Further, an apparatus or a component of an apparatus that is configured in a certain way is configured in at least that way, but it can also be configured in other ways than those specifically described.

The terms “comprise” (and any form of comprise, such as “comprises” and “comprising”), “have” (and any form of have, such as “has” and “having”), “include” (and any form of include, such as “includes” and “including”), and “contain” (and any form of contain, such as “contains” and “containing”) are open-ended linking verbs. As a result, an apparatus that “comprises,” “has,” “includes,” or “contains” one or more elements possesses those one or more elements, but is not limited to possessing only those elements. Likewise, a method that “comprises,” “has,” “includes,” or “contains” one or more steps possesses those one or more steps, but is not limited to possessing only those one or more steps. Additionally, terms such as “first” and “second” are used only to differentiate steps, structures, features, or the like, and not to limit the different structures or features to a particular order.

Any embodiment of any of the apparatuses and methods can consist of or consist essentially of—rather than comprise/include/contain/have—any of the described elements, features, and/or steps. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one embodiment may be applied to other embodiments, even though not described or illustrated, unless expressly prohibited by this disclosure or the nature of the embodiments.

Details associated with the embodiments described above and others are described below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers.

FIG. 1 is a perspective view of one embodiment of the present surgical apparatuses.

FIG. 2 is a perspective view of another embodiment of the present surgical apparatuses.

FIG. 3 is a perspective view of still another embodiment of the present surgical apparatuses, which has the same elongated member as the first embodiment but a different coupling structure.

FIG. 4 is a perspective view of yet another embodiment of the present surgical apparatuses.

FIG. 5 is a perspective view of one embodiment of the present surgical apparatuses coupled to an endoscope.

### DETAILED DESCRIPTION

Referring to FIG. 1, surgical apparatus 100 includes elongated member 130, a portion of which is shown in FIG. 1, and coupling structure 150, which is attached to or integral with elongated member 130. In this embodiment, elongated member 130 has an angled distal end 132 and first opening 134 is positioned at distal end 132. Elongated member 130 includes an inner surface 136 that defines channel 138, which extends between first opening 134 and a second opening (not shown in this figure). Axis 135 is centered in and runs within channel 138. While first opening 134 is located at distal end 132 of elongated member 130 in this embodiment, in other embodiments, the first opening may be located elsewhere, such as along the length of the elongated member at a location spaced apart from the distal end of the elongated member, which may be closed. For example, in some embodiments of the present elongated members (not shown), the distal end of the member may be closed and blunt (to minimize trauma on insertion), and one or more openings may be positioned near the closed distal end or at any desired locations proximal from the distal end (like fenestrations), including at spaced-apart locations along a portion of the elongated member. Elongated member 130 is shown in this embodiment as having a tubular shape, and therefore a circular cross-sectional profile taken perpendicular to axis 135. But in other embodiments, elongated member 130 may have any other shape suited to a given application, such as oval.

In most embodiments, elongated member 130 will have a consistent cross-sectional profile, and may comprise surgical tubing. The size of it may be suited to the application. The material from which elongated member 130 is made should be thin and soft enough to not cause excessive trauma on insertion into the body, yet strong enough that it does not collapse under suction. In some embodiments, at least one location along the length elongated member 130 will have an inner cross sectional area of at least 0.12 centimeters squared (cm²) (including at least 0.20 cm², at least 0.28 cm², at least 0.38 cm², at least 0.50 cm², at least 0.64 cm ², at least 0.79 cm², at least 0.95 cm², at least 1.13 cm², at least 1.33 cm², at least 1.54 cm², at least 1.77 cm², or more); in some embodiments, that inner cross-sectional area (which may be at least 0.28 cm²) will exist at every location that is completely bounded by the elongated member perpendicular to axis 135 at a given location along the axis (or length) of the elongated member (in contrast, such an area will not exist at locations bounded by the angled distal end of the elongated member). In other words, that inner cross-sectional area may exist at each location along the channel at which a plane (not shown) intersecting axis 135 also intersects inner surface 136 in a closed perimeter. For example, elongated member 130 may have a tubular shape and channel 138 may have an inner diameter of at least 4 millimeters (including 5 and 5.5 mm). As another example, elongated member 130 may have a tubular shape and channel 138 may have an inner diameter of at least 6 mm (including 6.5, 7, 7.5, 8, 8.5, 9, 9.5, and 10 mm or more). Such channel sizes may be used to be used to remove or introduce material at a greater rate and/or of a larger size than is possible by some channels located within some endoscopes.

Coupling structure 150 is configured to couple elongated member 130 to an endoscope (not shown). In the embodiment shown in FIG. 1, coupling structure 150 comprises a cap that is configured to fit over a distal end of an endoscope. The cap includes an opening (specifically, a distal opening) 152 that is sized so that none of the functionality of (and, more specifically, none of the functional components located at) the distal end of the endoscope (including a lens and any channels for instruments, suction, and/or air) is impeded. Opening 152 is bordered by rim 154 that is configured to abut against a portion of the distal end of an endoscope so that the endoscope interferes with the cap and the cap cannot move proximally along the endoscope. The cap is open at its proximal end as well, allowing it to be slipped over the end of an endoscope. The cap may alternatively be configured with one or more hinges (such as a living hinge) that allow it to be clamped about the distal end of an endoscope. The cap may have any suitable inner cross-sectional area for at least a portion of its length, including an inner cross-sectional area of at least 0.12 cm² or more, including at least 0.20 cm², at least 0.28 cm², at least 0.38 cm², at least 0.50 cm², at least 0.64 cm², at least 0.79 cm², at least 0.95 cm², at least 1.13 cm², at least 1.33 cm², at least 1.54 cm², at least 1.77 cm², or more. The cap can be made from any suitable medical-grade material and can be configured to fit tightly over the end of an endoscope so that it stays in place during a procedure as the possibility that it will otherwise slip off is minimized. Examples of caps that could be modified to be suitable for use as the present caps include the polarizing filter cap that is configured for use with the Third Eye® Retroscope® device (Avantis), and variceal ligator caps.

Some embodiments of coupling structure 150 can be attached to elongated member 130 in any suitable fashion, including through the use of an adhesive or through sonic welding. Alternatively, coupling structure 150 may be formed integrally with elongated member 130. For example, coupling structure 150 and elongated member 130 may be formed of rigid plastic, metal, or other suitable material, and molded or forged as one piece, such as through injection molding or the like. The attachment between or integration of coupling structure 150 and elongated member 130 may create a fixed relationship between coupling structure 150 and elongated member 130 such that a fixed position and orientation of distal end 132 of elongated member 130 with respect to the distal end of an endoscope may be predetermined.

Versions of coupling structure 150 other than caps may be used. For example, coupling structure 150 may comprise a clip structure having back-to-back openings for receiving an endoscope in one clip-shaped opening and an elongated member in the other clip-shaped opening, or a clamping structure, such as one that includes back-to-back clamps, one sized for an endoscope and another for an elongated member.

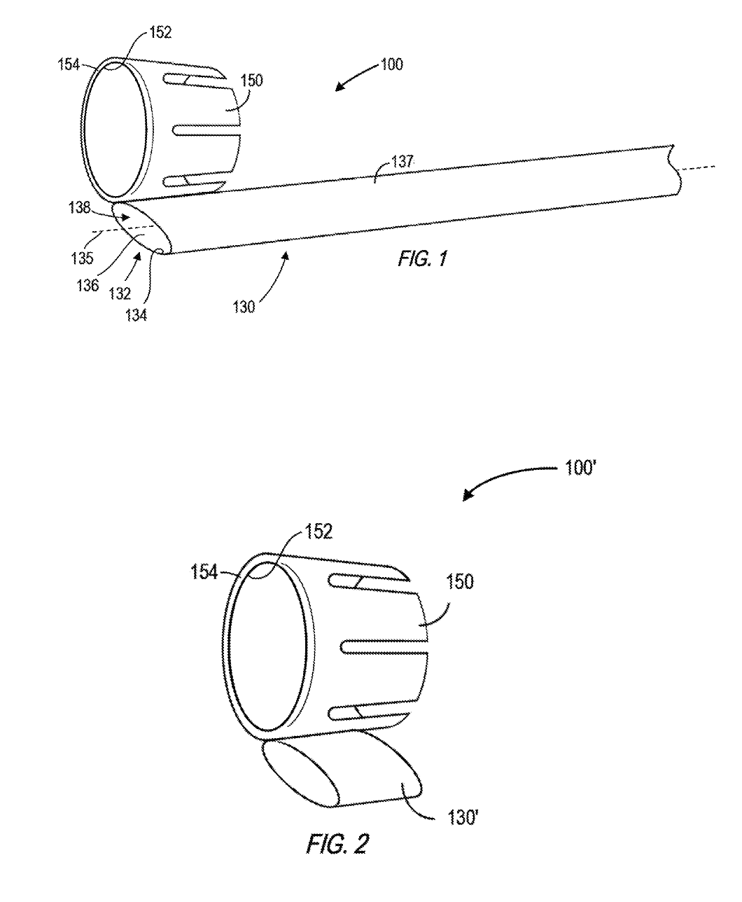

In an alternative embodiment to surgical apparatus 100, surgical apparatus 100′ shown in FIG. 2 includes coupling structure 150 (in the form, e.g., of a cap) that is attached to or integral with an elongated member coupling structure 130′, which can take the form of a cap (with or without a rim, like rim 154) that is configured to fit over the end of an elongated member (such as surgical tubing) or a sleeve (like coupling structure 250, discussed below) through which an elongated member may be positioned, provided the sleeve possesses sufficient elasticity and/or a surface that creates sufficient friction with the elongated member that the two will not move with respect to each other during placement, removal, or otherwise during the procedure itself.

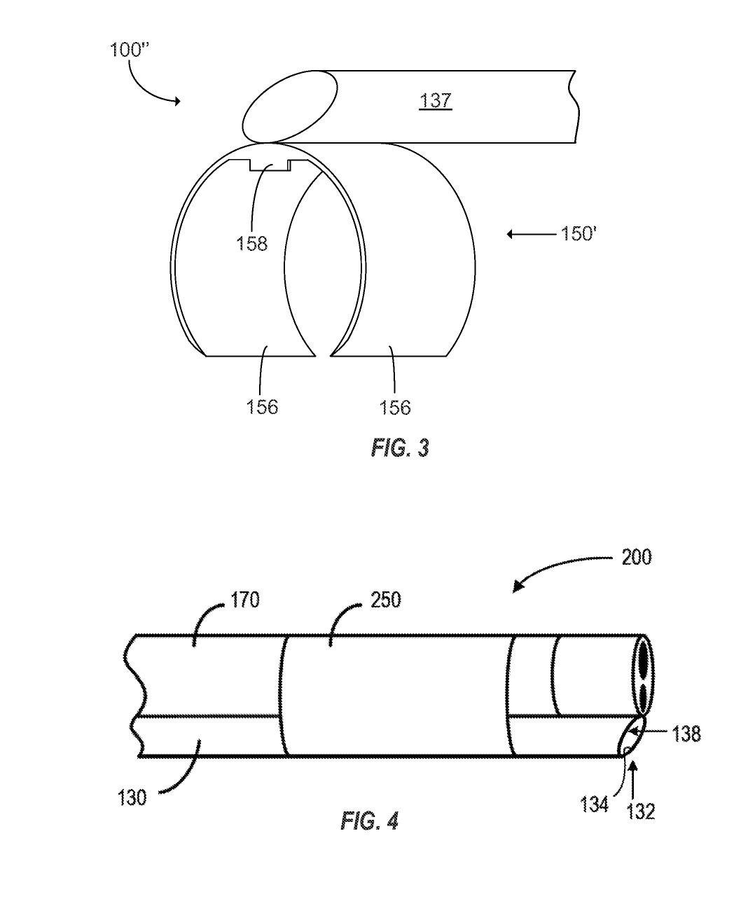

FIG. 3 shows surgical apparatus 100”, an alternative embodiment to surgical apparatus 100 that includes elongated member 130 and coupling structure 150′, which is attached to or integral with elongate member 130 and configured to couple elongated member 130 to an endoscope (not shown). Coupling structure 150′ is another embodiment of a cap that is configured to fit over a distal end of an endoscope. In the depicted embodiment, the cap of coupling structure 150′ is configured with two curved portions 156 that are configured to at least partially surround an endoscope. Curved portions 156 extend toward but do not touch each other. Coupling structure 150′ is an example of a coupling structure (and, more specifically, of a cap) that is configured (e.g., via curved portions 156) to not completely surround (or encircle) a structure (such as a tubular structure, like an endoscope). Coupling structure 150′ has the shape of a portion of a tube, and is open at both of its ends and it is longitudinally open (between its ends) because curved portions 156 do not touch each other. Coupling structure 150′ is an example of a coupling structure (and, more specifically, of a cap) that is configured (e.g., via curved portions 156) to be coupled to a structure (such as a tubular structure, like an endoscope) in multiple ways, including by (1) sliding it over the end of the structure, and, alternatively, (2) by pushing the structure laterally (or sideways) into the gap defined by the curved portions, and by temporarily displacing the curved portions in the process (which, due to their at least partially resilient construction, return to substantially their non-displaced positions). Coupling structure 150′ includes (e.g., is configured with) a portion (portion 158, which, in this embodiment, is in the form of a tab projecting toward the center of coupling structure 150′) configured to abut against a portion of the distal end of an endoscope so that the endoscope interferes with the cap and the cap cannot move proximally along the endoscope. Coupling structure 150′ may have any suitable inner cross-sectional area for at least a portion of its length, including an inner cross-sectional area of at least 0.12 cm² or more, including at least 0.20 cm², at least 0.28 cm², at least 0.38 cm², at least 0.50 cm², at least 0.64 cm², at least 0.79 cm², at least 0.95 cm², at least 1.13 cm², at least 1.33 cm², at least 1.54 cm², at least 1.77 cm², or more. Coupling structure 150′ made from any suitable medical-grade material and can be configured to fit tightly over the end of an (or at least partially around an) endoscope so that it stays in place during a procedure as the possibility that it will otherwise slip off is minimized.

FIG. 4 shows a partial perspective view of surgical apparatus 200, another embodiment of the present surgical apparatuses, coupled to endoscope 170. Surgical apparatus 200 includes coupling structure 250, which comprises a flexible sleeve, and elongated member 130. Coupling structure 250 is configured to couple elongated member 130 to an endoscope, and is shown in FIG. 4 as doing that. Specifically, as shown in FIG. 4, coupling structure 250 has been positioned along the exteriors of both elongated member 130 and endoscope 170 and couples the two together, with the distal ends of the endoscope and the elongated member being aligned in the depicted embodiment. The flexible sleeve of coupling structure 250 may initially have a rolled-up form; the rolled-up sleeve can then be positioned at a desired location and then unrolled proximally along the endoscope and the elongated member to couple them together. The material from which coupling structure 250 may be formed may be latex free and may have some elasticity in some embodiments, and in other embodiments, it may not have elasticity.

Coupling structure 250 may have any suitable length. In some embodiments, the length of coupling structure 250 may be 50 percent or less of the length of elongated member 130 (including 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 percent or less, but greater than 0 percent of the length of elongated member 130). In other embodiments, the length of coupling structure 250 may be less than 100 percent but greater than 50 percent of the length of elongated member 130 (including less than 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51 but greater than 50 percent of the length of elongated member 130).

Surgical apparatus 200 may include multiple coupling structures 250 that can be positioned at multiple locations along the respective lengths of elongated member 130 and endoscope 170. Coupling structure 250 may be configured so that, when positioned around both endoscope 170 and elongated member 130, coupling structure 250 does not overly compress channel 138 of elongated member 130. With coupling structure 250, distal end 132 of elongated member 130 may be positioned in any desired location with respect to the distal end of the endoscope to which the elongated member is coupled. Thus, the first opening of channel 138 may be positioned distal or proximal of the distal end of the endoscope. In addition, the use of coupling structure 250 may afford the surgeon with the option of cutting distal end 132 (and, in some embodiments, therefore, first opening 134) of elongated member 130 to any desired angle, curve, or shape that may be desired to address the needs of a particular patient or procedure.

For embodiments of coupling structure 250 that possess a sufficient degree of elasticity, it may be possible to use one size of coupling structure 250 to function with most or all available endoscopes. Coupling structure 150, when configured as a cap, may require more size varieties to function with the same range of endoscopes.

Embodiments of the present surgical apparatuses (e.g., surgical apparatuses 100, 100′, and 200) may also include one or more additional coupling structures, for further coupling elongated member 130 to an endoscope at locations proximal to the location at which coupling structure 150 or 250 is used (in some embodiments, coupling structures 150 and 250 can be part of the same embodiment and, thus, both used to couple elongated member 130 to an endoscope, and multiple coupling structures 250 may be included in some embodiments of the present surgical apparatuses).

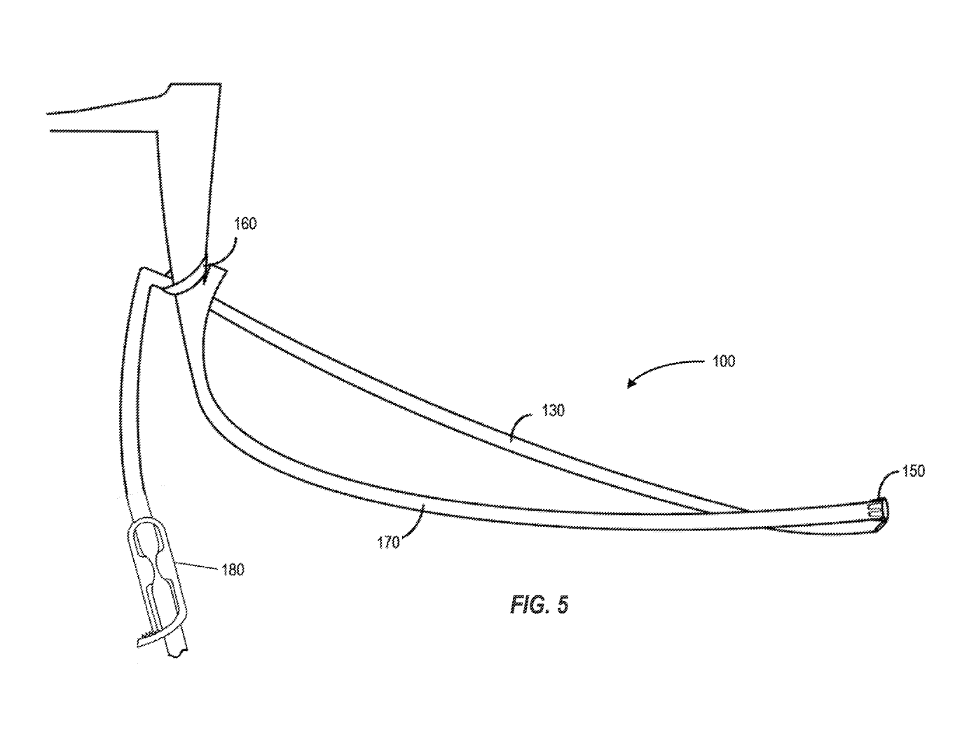

For example, FIG. 5 shows a perspective view of surgical apparatus 100 in which elongated member 130 has been coupled to endoscope 170 using coupling structure 150 and further using coupling structure 160, which comprises at least one plastic tie—such as a zip tie, cable tie, or other suitable structure configured to be positioned around (e.g., in a loop) both endoscope 170 and elongated member 130. As shown, coupling structure 160 has been used to couple endoscope 170 to elongated member 130 at a location that is proximal to the location at which coupling structure 150 couples endoscope 170 to elongated member 130. Such additional coupling structures 160 may be employed at multiple locations along the lengths of elongated member 130 and endoscope 170, and may form part of surgical apparatus 100.

Embodiments of the present surgical apparatuses (e.g., surgical apparatuses 100, 100′, and 200) may also include a valve member, such as clamp 180 shown in FIG. 4, which may be actuated to preclude the flow of material (including air) through elongated member 130 in either direction.

Embodiments of the present surgical apparatuses may take the form of kits that include a container (e.g., a tray (e.g., a sealed tray), a box, or a pouch (e.g., a sealed, flexible pouch)) in which the components of one of the present surgical apparatuses (e.g., surgical apparatus 100, 100′, or 200) are disposed and that also includes instructions for use (e.g., on the outside of the container (e.g., on a sticker) or on material disposed inside the container (e.g., a written insert) with the surgical apparatus components). One or more of the components of the surgical apparatus included in the container may be sterile.

This disclosure also includes methods, which may be characterized as medical procedure-related methods. Some embodiments of the present methods comprise a method of instructing one or more persons about how to use one of the present surgical apparatuses. The instruction may occur in person live, in person via video, or pre-recorded. The method may include inserting an endoscope coupled to an elongated member (in any manner disclosed herein) into any location (e.g., a lumen, like a body cavity, or extra-lumen, like the pleural cavity or the peritoneum) of a subject (e.g., a human patient), including a stomach, colon, intestine, bladder, bronchus, ear, nose, throat, or the like. For example, the method may include coupling an elongated member having a distal end to an endoscope having a distal end so that the distal ends of the elongated member and of the endoscope are aligned. In other embodiments, the method may include coupling an elongated member having a length and a channel to an endoscope having a length so that: the elongated member is positioned beside the endoscope for a portion of their respective lengths, no portion of the elongated member is coaxial with any portion of the endoscope, and no portion of the channel of the elongated member is in communication with any channel of the endoscope. With any of these embodiments, the elongated member has a channel that extends between a first opening in the elongated member and a second opening in the elongated member, and the channel has an inner cross sectional area of at least 0.12 cm2 at at least one location taken perpendicular to an axis centered in and running within the channel. The endoscope that is used may be suited and sized for any one of more of a variety of applications, including, but not limited to, urology (cystoscopy); pulmonary applications (bronchoscopy); ear, nose and throat applications; SILS (single incision laparoscopic surgery), NOTES (natural orifice transluminal endoscopic surgery), or even “extra-lumen” (pleura, peritoneum).

The coupling may be accomplished using at least a coupling structure, like coupling structure 150, to which the elongated member is coupled (e.g., via elongated member coupling structure 130′) and to which the endoscope is coupled. Alternatively, the coupling may be accomplished using at least a coupling structure (e.g., coupling structure 150) to which the elongated member is attached or with which the elongated member is integral. Still further, the coupling may be accomplished using at least a flexible sleeve.

Embodiments of the present methods may also include using at least another coupling structure to further couple the elongated member to the endoscope at a location proximal of the first coupling structure, such as coupling structure 160. One or more such coupling structures may be used for this purpose.

Embodiments of the present methods may also include locating a valve member at a location distal of the proximal end of the elongated member, such as clamp 180. Embodiments of the present methods may also include regulating the flow of material through the elongated member by actuating the valve member.

Embodiments of the present methods may also include coupling the elongated member to a suction canister and, following introduction of the elongated member and endoscope into an animal (e.g., a human, such as a human patient), removing material from at least one of a stomach, an intestine, and a colon of the animal. In such a circumstance, a valve member like clamp 180 may be actuated to allow flow through the channel, and thus effect removal of contents from the stomach or colon of the body of the patient or subject via suction. For example, blood clots and/or partially digested food may be removed in this fashion.

Embodiments of the present methods may also include coupling the elongated member to at least one of a syringe and a pump and, following introduction of the elongated member and endoscope into an animal (e.g., a human, such as a human patient), introducing material into the animal. In such a circumstance, a valve member like clamp 180 may be actuated to allow flow of contents through the channel, and the syringe or pump may be operated to introduce the contents to the stomach, an intestine, or colon of the body of the patient or subject. The contents may include, but are not limited to, fecal microbiota transplant.

Embodiments of the present methods may also include inserting the endoscope and the elongated member into an animal, such as into the stomach or colon of a human patient. During such insertion, flow may be restricted through the elongated member, such as using a valve member like clamp 180.

The above specification and examples provide a complete description of the structure and use of illustrative embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the devices are not intended to be limited to the particular foci s disclosed. Rather, they include all modifications and alternatives falling within the scope of the claims, and embodiments other than the one shown may include some or all of the features of the depicted embodiments. For example, while coupling structure 250 is shown as having only one lumen in which both an endoscope and an elongated member may be positioned, in other embodiments (not shown), coupling structure 250 may have an inner divider that creates two lumens, one for the endoscope and one for the elongated member, and those separate lumens may be configured to fit snugly around those respective structures in a non-sliding fashion when used. Where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. A surgical apparatus comprising:
an elongated member having a channel that extends between a first opening in the elongated member and a second opening in the elongated member, the elongated member having a distal end, the channel having an inner cross sectional area of at least 0.12 centimeters squared (cm²) at at least one location taken perpendicular to an axis centered in and running within the channel; and
a coupling structure attached to or integral with the elongated member and configured to couple the elongated member to an endoscope.

2. The surgical apparatus of claim 1, where the coupling structure includes curved portions that extend toward but do not touch each other.

3. The surgical apparatus of claim 1, where the coupling structure is configured to not completely surround a tubular structure when coupled to the tubular structure.

4. The surgical apparatus of claim 1, where the coupling structure comprises a cap adapted to fit over a distal end of an endoscope.

5. The surgical apparatus of claim 4, where the first opening of the elongated structure is located at the distal end of the elongated member.

6. The surgical apparatus of claim 5, where the second opening of the elongated structure is located at a proximal end of the elongated member.

7. The surgical apparatus of claim of any of claims 2-6, where an inner surface of the elongated member defines the channel, and each location along the channel at which a plane intersecting the axis also intersects the inner surface in a closed perimeter has an inner cross sectional area of at least 0.12 cm².

8. The surgical apparatus of claim 7, where each location along the channel at which a plane intersecting the axis also intersects the inner surface in a closed perimeter has an inner cross sectional area of at least 0.28 cm².

9. The surgical apparatus of any of claims 2-7, where the distal end of the elongated member is attached to the cap by an adhesive.

10. The surgical apparatus of any of claims 2-9, further comprising:
another coupling structure configured to couple the elongated member to the endoscope at a location spaced apart from a distal end of the endoscope.

11. The surgical apparatus of claim 10, where the other coupling structure is at least one plastic tie adapted to form a loop about both the endoscope and the elongated member.

12. The surgical apparatus of claim 2, where the inner cross sectional area is at least 0.28 cm.

13. The surgical apparatus of claim 1, where the coupling structure is integral with the elongated member.

14. The surgical apparatus of claim 13, where the coupling structure comprises a cap adapted to fit over a distal end of an endoscope.

15. The surgical apparatus of claim 14, where the first opening of the elongated structure is located at the distal end of the elongated member.

16. The surgical apparatus of claim 15, where the second opening of the elongated structure is located at a proximal end of the elongated member.

17. The surgical apparatus of claim of any of claims 14-16, where an inner surface of the elongated member defines the channel, and each location along the channel at which a plane intersecting the axis also intersects the inner surface in a closed perimeter has an inner cross sectional area of at least 0.12 cm².

18. The surgical apparatus of claim 17, where each location along the channel at which a plane intersecting the axis also intersects the inner surface in a closed perimeter has an inner cross sectional area of at least 0.28 cm².

19. The surgical apparatus of any of claims 14-18, further comprising:
another coupling structure configured to couple the elongated member to the endoscope at a location spaced apart from a distal end of the endoscope.

20. The surgical apparatus of claim 19, where the other coupling structure is at least one plastic tie adapted to form a loop about both the endoscope and the elongated member.

21. A surgical apparatus comprising:
an elongated member having a channel that extends between a first opening in the elongated member and a second opening in the elongated member, the elongated member having a distal end, the channel having an inner cross sectional area of at least 0.12 centimeters squared (cm²) at at least one location taken perpendicular to an axis centered in and running within the channel; and
a flexible sleeve configured to couple the elongated member to an endoscope.

22. The surgical apparatus of claim 21, where the first opening of the elongated structure is located at the distal end of the elongated member.

23. The surgical apparatus of claim 22, where the second opening of the elongated structure is located at a proximal end of the elongated member.

24. The surgical apparatus of claim of any of claims 22-23, where an inner surface of the elongated member defines the channel, and each location along the channel at which a plane intersecting the axis also intersects the inner surface in a closed perimeter has an inner cross sectional area of at least 0.12 cm².

25. The surgical apparatus of claim 24, where each location along the channel at which a plane intersecting the axis also intersects the inner surface in a closed perimeter has an inner cross sectional area of at least 0.28 cm².

26. The surgical apparatus of any of claims 22-25, further comprising:
another coupling structure configured to couple the elongated member to the endoscope at a location spaced apart from a distal end of the endoscope.

27. The surgical apparatus of claim 26, where the other coupling structure is at least one plastic tie adapted to form a loop about both the endoscope and the elongated member.

28. The surgical apparatus of claim 21, where the inner cross sectional area is at least 0.28 cm.

29. A surgical apparatus comprising:
an elongated member coupling structure configured to be coupled to and surround an exterior portion of an elongated member; and
a coupling structure attached to or integral with the elongated member coupling structure and configured to couple the elongated member coupling structure to an endoscope.

30. The surgical apparatus of claim 29, where the coupling structure includes curved portions that extend toward but do not touch each other.

31. The surgical apparatus of claim 29, where the coupling structure is configured to not completely surround a tubular structure when coupled to the tubular structure.

32. The surgical apparatus of claim 29, where the coupling structure comprises a cap adapted to fit over a distal end of an endoscope.

33. The surgical apparatus of any of claims 29-32, further comprising:
another coupling structure configured to couple the elongated member to the endoscope at a location spaced apart from a distal end of the endoscope.

34. The surgical apparatus of claim 33, where the other coupling structure is at least one plastic tie adapted to form a loop about both the endoscope and the elongated member.

35. A medical procedure-related method comprising:
coupling an elongated member having a length and a channel to an endoscope having a length so that:
the elongated member is positioned beside the endoscope for a portion of their respective lengths,
no portion of the elongated member is coaxial with any portion of the endoscope, and
no portion of the channel of the elongated member is in communication with any channel of the endoscope;
where the channel extends between a first opening in the elongated member and a second opening in the elongated member; and
where the channel has an inner cross sectional area of at least 0.12 centimeters squared (cm²) at at least one location taken perpendicular to an axis centered in and running within the channel.

36. The medical procedure-related method of claim 35, where the coupling is accomplished using at least a coupling structure to which the elongated member is coupled and to which the endoscope is coupled.

37. The medical procedure-related method of claim 35, where the coupling is accomplished using at least a coupling structure to which the elongated member is attached or with which the elongated member is integral.

38. The medical procedure-related method of claim 37, where the first opening of the elongated member is located at the distal end of the elongated member.

39. The medical procedure-related method of claim 38, where the second opening of the elongated member is located at a proximal end of the elongated member.

40. The medical procedure-related method of any of claims 35-39, where an inner surface of the elongated member defines the channel, and each location along the channel at which a plane intersecting the axis also intersects the inner surface in a closed perimeter has an inner cross sectional area of at least 0.12 cm².

41. The medical procedure-related method of claim 40, where each location along the channel at which a plane intersecting the axis also intersects the inner surface in a closed perimeter has an inner cross sectional area of at least 0.28 cm².

42. The medical procedure-related method of any of claims 37-41, where the coupling structure comprises a cap adapted to fit over the distal end of the endoscope.

43. The medical procedure-related method of any of claims 37-42, further comprising:
inserting the endoscope and the elongated member into an animal.

44. The medical procedure-related method of claim 43, further comprising:
using the channel to at least one of introduce material into and remove material from the animal.

45. The medical procedure-related method of any of claims 37-42, where the coupling structure comprises a first coupling structure and the method further comprises:
using at least another coupling structure to further couple the elongated member to the endoscope at a location proximal of the first coupling structure. cm 46. The medical procedure-related method of claim 45, where the other coupling structure is at least one plastic tie, and the using the other coupling structure comprises wrapping the at least one plastic tie around both the endoscope and the elongated member.

47. The medical procedure-related method of claim 46, further comprising:
coupling the elongated member to a suction canister; and
removing material from at least one of a stomach and a colon of the animal.

48. The medical procedure-related method of claim 46, further comprising:
coupling the elongated member to at least one of a syringe and a pump; and
introducing material into the animal.

49. The medical procedure-related method of claim 35, where the coupling is accomplished using at least a flexible sleeve.

50. The medical procedure-related method of claim 49, where the first opening of the elongated member is located at the distal end of the elongated member.

51. The medical procedure-related method of claim 50, where the second opening of the elongated member is located at a proximal end of the elongated member.

52. The medical procedure-related method of any of claims 35 and 49-51, where an inner surface of the elongated member defines the channel, and each location along the channel at which a plane intersecting the axis also intersects the inner surface in a closed perimeter has an inner cross sectional area of at least 0.12 cm².

53. The medical procedure-related method of claim 52, where each location along the channel at which a plane intersecting the axis also intersects the inner surface in a closed perimeter has an inner cross sectional area of at least 0.28 cm².

54. The medical procedure-related method of any of claims 49-53, further comprising:
inserting the endoscope and the elongated member into an animal.

55. The medical procedure-related method of claim 54, further comprising:
using the channel to at least one of introduce material into and remove material from the animal.

56. The medical procedure-related method of any of claims 49-53, where the coupling structure comprises a first coupling structure and the method further comprises:
using at least another coupling structure to further couple the elongated member to the endoscope at a location proximal of the first coupling structure.

57. The medical procedure-related method of claim 56, where the other coupling structure is at least one plastic tie, and the using the other coupling structure comprises wrapping the at least one plastic tie around both the endoscope and the elongated member.

58. The medical procedure-related method of claim 57, further comprising:
coupling the elongated member to a suction canister; and
removing material from at least one of a stomach and a colon of the animal.

59. The medical procedure-related method of claim 57, further comprising:
coupling the elongated member to at least one of a syringe and a pump; and
introducing material into the animal.

60. A medical procedure-related method comprising:
coupling an elongated member having a distal end to an endoscope having a distal end so that the distal ends of the elongated member and of the endoscope are aligned;
where the elongated member has a channel that extends between a first opening in the elongated member and a second opening in the elongated member.

61. The medical procedure-related method of claim 60, where the channel has an inner cross sectional area of at least 0.12 centimeters squared (cm²) at at least one location taken perpendicular to an axis centered in and running within the channel.

62. The medical procedure-related method of any of claims 60-61, where the coupling is accomplished using at least a coupling structure to which the elongated member is coupled and to which the endoscope is coupled.

63. The medical procedure-related method of any of claims 60-61, where the coupling is accomplished using at least a coupling structure to which the elongated member is attached or with which the elongated member is integral.

64. The medical procedure-related method of claim 63, where the first opening of the elongated member is located at the distal end of the elongated member.

65. The medical procedure-related method of claim 64, where the second opening of the elongated member is located at a proximal end of the elongated member.

66. The medical procedure-related method of any of claims 60-65, where an inner surface of the elongated member defines the channel, and each location along the channel at which a plane intersecting the axis also intersects the inner surface in a closed perimeter has an inner cross sectional area of at least 0.12 cm².

67. The medical procedure-related method of claim 66, where each location along the channel at which a plane intersecting the axis also intersects the inner surface in a closed perimeter has an inner cross sectional area of at least 0.28 cm².

68. The medical procedure-related method of any of claims 63-67, where the coupling structure comprises a cap adapted to fit over the distal end of the endoscope.

69. The medical procedure-related method of any of claims 63-68, further comprising:
inserting the endoscope and the elongated member into an animal.

70. The medical procedure-related method of claim 69, further comprising:
using the channel to at least one of introduce material into and remove material from the animal.

71. The medical procedure-related method of any of claims 63-68, where the coupling structure comprises a first coupling structure and the method further comprises:
using at least another coupling structure to further couple the elongated member to the endoscope at a location proximal of the first coupling structure.

72. The medical procedure-related method of claim 71, where the other coupling structure is at least one plastic tie, and the using the other coupling structure comprises wrapping the at least one plastic tie around both the endoscope and the elongated member.

73. The medical procedure-related method of claim 72, further comprising:
coupling the elongated member to a suction canister; and
removing material from at least one of a stomach and a colon of the animal.

74. The medical procedure-related method of claim 72, further comprising:
coupling the elongated member to at least one of a syringe and a pump; and
introducing material into the animal.

75. The medical procedure-related method of claim 60, where the coupling is accomplished using at least a flexible sleeve.

76. The medical procedure-related method of claim 75, where the first opening of the elongated member is located at the distal end of the elongated member.

77. The medical procedure-related method of claim 76, where the second opening of the elongated member is located at a proximal end of the elongated member.

78. The medical procedure-related method of any of claims 60 and 75-77, where an inner surface of the elongated member defines the channel, and each location along the channel at which a plane intersecting the axis also intersects the inner surface in a closed perimeter has an inner cross sectional area of at least 0.12 cm².

79. The medical procedure-related method of claim 78, where each location along the channel at which a plane intersecting the axis also intersects the inner surface in a closed perimeter has an inner cross sectional area of at least 0.28 cm².

80. The medical procedure-related method of any of claims 75-79, further comprising:
inserting the endoscope and the elongated member into an animal.

81. The medical procedure-related method of claim 80, further comprising:
using the channel to at least one of introduce material into and remove material from the animal.

82. The medical procedure-related method of any of claims 75-79, where the coupling structure comprises a first coupling structure and the method further comprises:
using at least another coupling structure to further couple the elongated member to the endoscope at a location proximal of the first coupling structure.

83. The medical procedure-related method of claim 82, where the other coupling structure is at least one plastic tie, and the using the other coupling structure comprises wrapping the at least one plastic tie around both the endoscope and the elongated member.

84. The medical procedure-related method of claim 83, further comprising:
coupling the elongated member to a suction canister; and
removing material from at least one of a stomach and a colon of the animal.

85. The medical procedure-related method of claim 83, further comprising:
coupling the elongated member to at least one of a syringe and a pump; and
introducing material into the animal.
